(19) United States
(12) Patent Application Publication
(10) Pub. No.: US 2026/0110559 A1
(43) Pub. Date: Apr. 23, 2026

(54) FLUID ANALYZER ANTENNA MOUNTING

(71) Applicant: Phase Dynamics, Inc., Richardson, TX (US)

(72) Inventor: Bentley N. Scott, Garland, TX (US)

(73) Assignee: Phase Dynamics, Inc., Richardson, TX (US)

(21) Appl. No.: 18/922,336

(22) Filed: Oct. 21, 2024

(51) Int. Cl. G01D 11/30; G01N 33/18; H01Q 1/22

(52) U.S. Cl. CPC G01D 11/30; G01N 33/1833; H01Q 1/22

(57) ABSTRACT

An apparatus for mounting a fluid analyzer. The apparatus comprises a pipe segment configured to contain a flow of liquid. A portion of the pipe segment forms an opening in a wall of the pipe segment. A housing is disposed in an interior of the pipe segment, wherein the housing is rigidly mounted to the pipe segment proximate the opening in the wall of the pipe segment. The apparatus also include a circuit board disposed in the interior of the housing and an antenna coupled to the circuit board. The antenna is aligned coaxially with the center of the pipe segment.

### TECHNICAL FIELD

The present application relates generally to fluid analyzers for testing water in petroleum products and, more specifically to a water-in-oil fluid analyzer for oilfield applications such as pipelines and other carriers.

### BACKGROUND

Water-in-oil analyzers are commonly used in oilfield applications. A water-in-oil analyzer typically uses a frequency of 10,000 KHz to 20 MHz methods to determine the capacitance which is related to the relative percentages of water and oil in a pipeline filled with hydrocarbon fluid. A water-in-oil analyzer commonly includes a coaxial structure that transmits a signal into a 2″ to 4″ diameter pipe for analysis of the “water in oil” content. One difficulty of such equipment is supporting the structures for capacitive analyzers, which typically occupy the majority of the inner diameter to achieve the highest capacitance for sensitivity improvement. Methods to hold the coaxial structure (the Element Tube) require a dielectric support which can withstand vibration from the flowing fluids. As these supports age the measurement is severely compromised.

FIG. 4A illustrates a conventional coaxial structure for a capacitance analyzer according to an embodiment of the prior art. FIG. 4B illustrates a conventional coaxial structure for a capacitance analyzer according to a second embodiment of the prior art. In both embodiments, an Element Tube must be held fixed in place to prevent vibrations or turbulence from affecting the measurement. Since the Element Tube also must be isolated from the body of the pipe, the feed-through from the electronics supporting the center of the Element Tube has a Teflon (PTFE) insulator and a small metal shaft which is connected to the sensor. Each end of the Element Tube is prevented from vibrating or moving out of center by the supports on each end. These supports must be replaced often.

Structures like these are disclosed in U.S. Pat. Nos. 4,751,842, 2,720,624, and 5,263,363. The disclosures of U.S. Pat. Nos. 4,751,842, 2,720,624, and 5,263,363 are hereby incorporated by reference into the present disclosure as if fully set forth herein. In RF and microwave analyzers, the antennas may be mounted such that the length is short to prevent any turbulence effects on measurements. Some antennas may be vertically mounted across the pipe to prevent vibrations, although these may be less effective since the ground plane or cage must be incorporated in the vertical section.

FIG. 4C illustrates a ground cage mounted in a pipe section according to an embodiment of the prior art. The ground cage is necessary to contain the electromagnetic field with antennas inside. This configuration may limit the area of measurement to the area inside the small ground cage. A similar embodiment is shown in U.S. Pat. No. 9,109,995. The disclosure of U.S. Pat. No. 9,109,995 is hereby incorporated by reference into the present disclosure as if fully set forth herein.

FIG. 4D illustrates a configuration for a microwave solution according to an embodiment of the prior art. The configuration includes two probes which are the transmit and receive antennas with a Fin structure to maintain the field pattern. This structure limits the area of measurement to the region where the Fin structure is located and limits the range of microwave frequencies available. A similar embodiment is shown in U.S. Patent Publication No. 2018-0045662. The disclosure of U.S. Patent Publication No. 2018-0045662 is hereby incorporated by reference into the present disclosure as if fully set forth herein.

Another probe type antenna system is described in FIGS. 3 and 4 of U.S. Pat. No. 5,101,163. The disclosure of U.S. Pat. No. 5,101,163 is hereby incorporated by reference into the present disclosure as if fully set forth herein. The disclosed probes are approximately 0.5″ ceramic caps protruding only a very short distance into the pipe. The frequency range and area of measurement were compromised by the locations and limited regions of measurement.

FIG. 4E illustrates an antenna that is aligned coaxially along the length of pipe according to an embodiment of the prior art. Measurement are improved due to a longer antenna and a full cross-sectional area of measurement in the pipe. This makes the frequency range available very wide. But the installation is more difficult as it must be an L-shaped pipe section instead of a straight pipe run. The same structure could be used in a U-shaped pipe section but this further limits the ability to install in a straight run of pipe.

### SUMMARY

It is a primary object of the present disclosure to provide an apparatus for mounting a water-in-oil fluid analyzer. The apparatus comprises: i) a pipe segment configured to contain a flow of liquid, wherein a portion of the pipe segment forms an opening in a wall of the pipe segment; ii) a housing disposed in an interior of the pipe segment, wherein the housing is rigidly mounted to the pipe segment proximate the opening in the wall of the pipe segment; iii) a circuit board disposed in the interior of the housing; and iv) an antenna coupled to the circuit board, wherein the antenna is aligned coaxially with the center of the pipe segment.

In one embodiment, the housing comprises a metal material.

In another embodiment, the housing is rigidly mounted to the pipe segment by welding the housing to the pipe segment proximate the opening in the pipe segment.

In still another embodiment, the antenna comprises a ceramic sheath and a metal rod encased withing the ceramic sheath.

In yet another embodiment, the antenna consisting of a ceramic rod with a hole in the center for the metal antenna which is rigidly connected to the housing using a metal collar brazed to the ceramic rod configured to electrically couple the metal center rod antenna to the circuit board and to form a hermetic seal with the housing.

In a further embodiment, the apparatus further comprises a radio frequency (RF) connector that forms an electrical connection between the circuit board and an external device through the opening in the pipe segment.

In a still further embodiment, the opening in the pipe segment is located on a top surface of the pipe segment.

In a yet another embodiment, the opening in the pipe segment is located on a side surface of the pipe segment.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is an illustration of a system according to an embodiment of the present disclosure.

FIG. 2 is a transparent perspective view of the system in FIG. 1 according to an embodiment of the present disclosure.

FIG. 3 is an exploded view of the system in FIG. 3 according to an embodiment of the present disclosure.

FIG. 4A illustrates a conventional coaxial antennas for a capacitance analyzer according to an embodiment of the prior art.

FIG. 4B illustrates a conventional coaxial antenna for a capacitance analyzer according to a second embodiment of the prior art.

FIG. 4C illustrates a ground cage mounted in a pipe section according to an embodiment of the prior art.

FIG. 4D illustrates a configuration for a microwave solution according to an embodiment of the prior art.

FIG. 4E illustrates an antenna that is aligned coaxially along the length of pipe according to an embodiment of the prior art.

### DETAILED DESCRIPTION

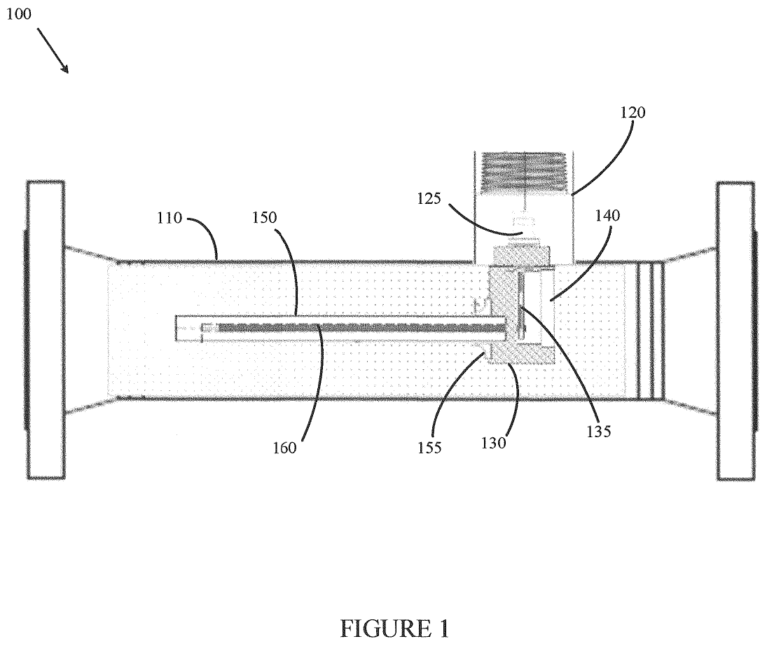

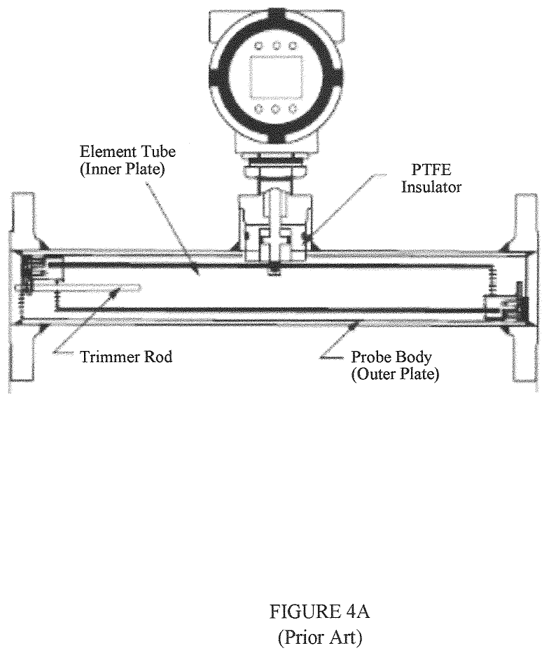

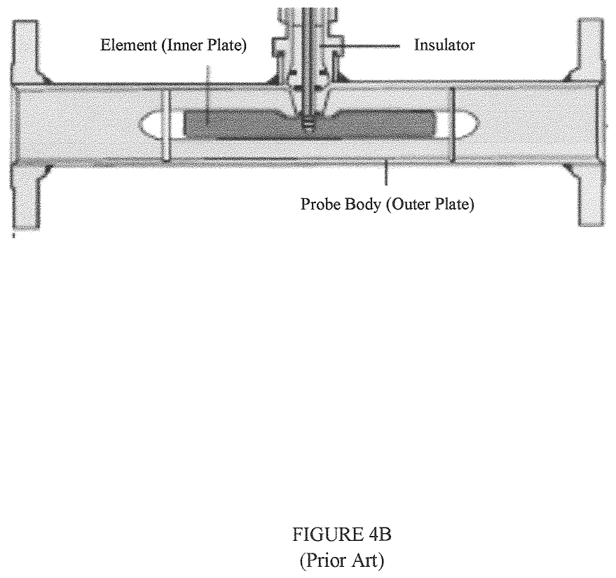

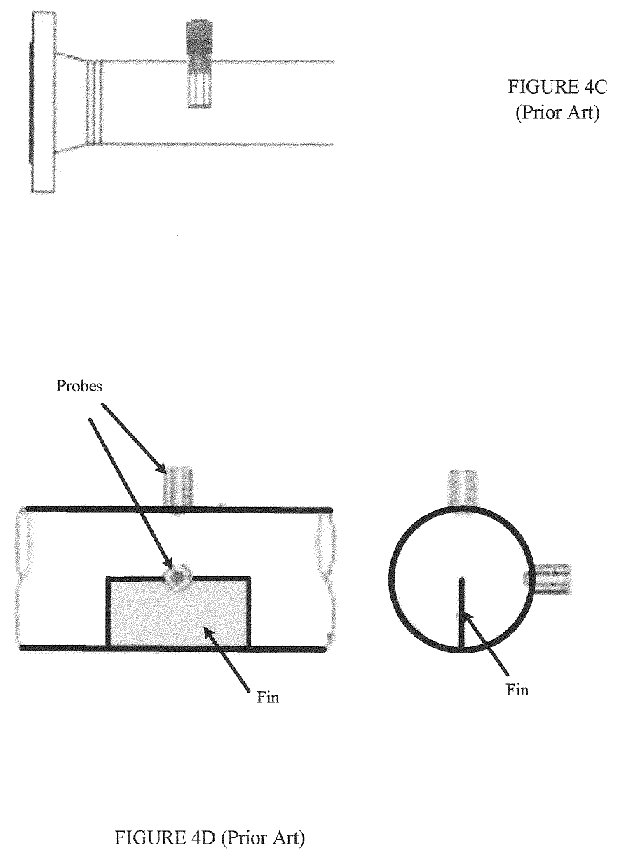

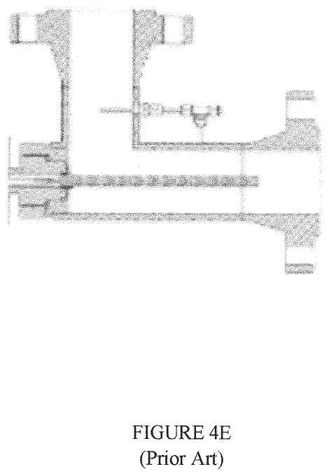

FIGS. 1 through 4E, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged fluid analyzer.

The present disclosure describes a novel fully rigid mounting and connection system for injecting a microwave signal into an oilfield pipe for the purpose of measuring water-in-oil. The disclosed apparatus comprises corrosion resistant metals coupled with a fully welded and brazed ceramic antenna. The apparatus suppresses vibrations due to turbulence caused by the petroleum products and water flows. The apparatus also includes additional matching network components to make a 90-degree transition of the electromagnetic signal from the transmitter mounted outside of the pipe to the antenna structure inside the pipe. The resulting system is fully welded and supported from the exterior of the pipe and sealed.

This disclosure provides a solution to the problems described above using an internal metal housing welded to an opening located at the top of the exterior of the pipe that supports the entire structure. In an embodiment, the antenna may comprise a metal rod encased in a solid ceramic sheath made of aluminum oxide. The apparatus is a two dielectric coaxial line solution. One dielectric is the ceramic rod with a dielectric of 9. The second is the flowing liquid medium, which is typically oil, at a dielectric of 2.5. The ceramic is brazed to a stainless-steel flange, which is then welded to the metal housing. This rigid structure prevents any turbulence or vibration from affecting its position within the coaxial structure, thereby maintaining an excellent measurement of the water percentage.

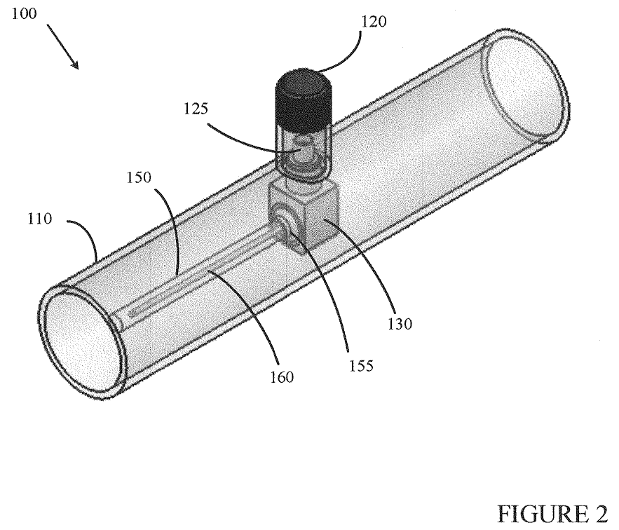

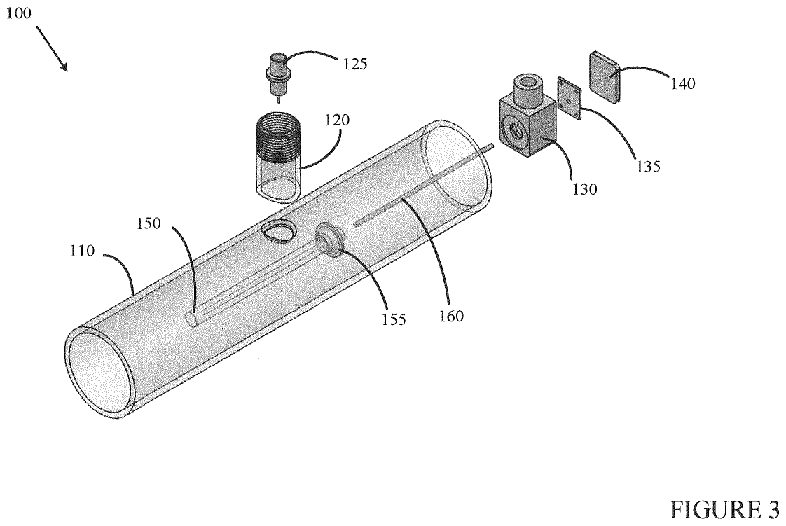

FIG. 1 is an illustration of a system 100 according to an embodiment of the present disclosure. FIG. 2 is a transparent perspective view of system 100 according to an embodiment of the present disclosure. FIG. 3 is an exploded view of system 100 according to an embodiment of the present disclosure.

The system 100 includes a flanged pipe segment 110 in which a liquid medium (dotted pattern) flows. In an embodiment, the liquid medium may be a hydrocarbon product that contains a mix of oil, water, and entrained gas. The system 100 also includes a threaded pipe 120 that is welded onto the top of pipe segment 110 proximate a circular opening in a sidewall of pipe segment 110. The threaded pip 120 provides a mounting for external transmitters and measurement sensors that may be part of the water-in-oil analyzer. The system 100 includes a metal housing 130 that forms an internal cavity that contains a circuit board 135.

The circuit board is coupled to an antenna rod 160 that is encased in a ceramic rod 150. The antenna rod 160 may comprise a brass rod. A ceramic collar 155 couples the ceramic rod and antenna rod 160 to the circuit board 135. The metal housing 130 is sealed by a back cover 140. In an embodiment, the back cover 140 may comprise a metal plate that is sealed by welding it to metal housing 130. Within pipe 120, an RF connector 125 is provided to provide an electrical connection to any matching circuitry on circuit board 130 and to external transceivers of a water-in-oil analyzer.

According to the principles of the present disclosure, the metal housing 130 includes an interior space or chamber to support circuit board 135 and for making the right-angle transition to the antenna rod 160. This provides an enclosure to make the 90-degree change in electromagnetic transition and supply any necessary matching network. The back cover 140 may be sealed by welding to the opening in metal housing 130 once the mechanical parts are placed properly into the cavity.

The transition of the electromagnetic field is made through the circuit board 135 to which the metal antenna may be soldered, mounted on the inside of the metal housing 130, and then screwed down. The upper RF connector 125 to the circuit board 135 includes a glass hermetic seal and therefore provides a secondary seal.

The disclosed straight two flanged pipe connection provides an easy entry of the analyzer into a pipeline in typical oil field applications, while providing an excellent transition into the flowing medium at single or multiple decade frequencies.

Although housing 130 may comprise a metal material, such as steel, this is by way of example only. In other embodiments, housing 130 may comprise other materials, such as plastic or ceramic. Additionally, housing 130 may be rigidly attached to pipe by means other than welding, such as by epoxy or another adhesive. Furthermore, the opening in pipe 110 is not required to be on the top surface of pipe segment 110. In alternate embodiment, the opening may be located on the sides or the bottom of pipe segment 110.

Although the present disclosure has been described with an exemplary embodiment, various changes and modifications may be suggested to one skilled in the art. It is intended that the present disclosure encompass such changes and modifications as fall within the scope of the appended claims.

What is claimed IS:

1. An apparatus for mounting a fluid analyzer including:
a pipe segment configured to contain a flow of liquid, wherein a portion of the pipe segment forms an opening in a wall of the pipe segment;
a housing disposed in an interior of the pipe segment, wherein the housing is rigidly mounted to the pipe segment proximate the opening in the wall of the pipe segment;
a circuit board disposed in the interior of the housing; and
an antenna coupled to the circuit board, wherein the antenna is aligned coaxially with the center of the pipe segment.

2. The apparatus as set forth in claim 1, wherein the housing comprises a metal material.

3. The apparatus as set forth in claim 2, wherein the housing is rigidly mounted to the pipe segment by welding the housing to the pipe segment proximate the opening in the pipe segment.

4. The apparatus as set forth in claim 1, wherein the antenna comprises a ceramic sheath and a metal rod encased within the ceramic sheath.

5. The apparatus as set forth in claim 4, wherein the antenna is rigidly connected to the housing using a metal collar brazed to the ceramic and configured to electrically couple the antenna to the circuit board and to form a hermetic seal with the housing.

6. The apparatus as set forth in claim 1, further including a radio frequency (RF) connector that forms an electrical connection between the circuit board and an external device through the opening in the pipe segment.

7. The apparatus as set forth in claim 6, wherein the opening in the pipe segment is located on a top surface of the pipe segment.

8. The apparatus as set forth in claim 6, wherein the opening in the pipe segment is located on a side surface of the pipe segment.
